(19) United States
(12) Patent Application Publication
(10) Pub. No.: US 2003/0022252 A1
(43) Pub. Date: Jan. 30, 2003

(54) Method for identifying tolerance in graft recipients

(76) Inventor: Angus W. Thomson, Pittsburgh, PA (US)

Correspondence Address: KIRKPATRICK & LOCKHART LLP, 535 SMITHFIELD STREET, PITTSBURGH, PA, 15222, US

(21) Appl. No.: 10/196,858

(22) Filed: Jul. 15, 2002

Related U.S. Application Data
(60) Provisional application No. 60/305,046, filed on Jul. 13, 2001.

(51) Int. Cl. G01N 33/567

(52) U.S. Cl. 435/7.21

(57) ABSTRACT

Provided is a method of identifying tolerance in allograft recipients. It has been found that higher levels of circulating plasmacytoid DC (pDC2 cells), as compared to monocytoid (pDC1) cells (pDC2/pDC1) correlates with transplant tolerance. In one embodiment, the method includes determining the relative amounts of HLA-DR⁺lin⁻(CD3⁻CD⁻14⁻CD19⁻CD20⁻)CD123⁺CD11c⁻(pDC2) cells and HLA-DR⁺lin⁻CD123^{lo}CD11c⁺(pDC1) cells in peripheral blood. The pDC1/pDC2 either can be determined from direct analysis of cells in a peripheral blood sample, or DC1 and DC2 cells can be cultured from a peripheral blood sample and the numbers of pDC1 and pDC2 cells in the peripheral blood sample can be estimated from the growth of the DC1 and DC2 cells in culture.

### CROSS REFERENCE TO RELATED APPLICATIONS

This application claims priority under 35 U.S.C. §119(e) to U.S. Provisional Patent Application No. 60/305,046, filed Jul. 13, 2001, which is incorporated herein by reference in its entirety.

### STATEMENT REGARDING FEDERAL FUNDING

This invention was made with government support under Grant Nos. DK 49745, DK 29961 and Al 95830, awarded by the National Institutes of Health. The government has certain rights in this invention.

### BACKGROUND

1. Field of the Invention

A method of identifying tolerance in a graft recipient, for example in an allograft liver transplant recipient.

2. Description of the Related Art

While it has been possible using immunosuppressive agents to induce organ transplant tolerance predictably in experimental animals for the past 50 years, no such protocols exist in humans. On the other hand, there are well-documented instances of withdrawal of immunosuppression from human transplant recipients without occurrence of graft dysfunction/rejection. Drug withdrawal may be required for the management of post-transplant viral infection (Epstein Barr virus [EBV], cytomegalovirus [CMV], human immunodeficiency virus, hepatitis B or C), post-transplant lymphoproliferative disease (PTLD), or may be the result of patient non-compliance, or physician-controlled protocolized weaning. While such instances of “operational tolerance” (defined herein as stable graft function, in the absence of immunosuppressive therapy) are very rare in kidney recipients, this is a more common finding following liver transplantation. At present, no immunologic or immunogenetic laboratory assays can predict or validate donor-specific tolerance in organ transplantation.

The immune response to organ grafts is believed to be initiated by the presentation of alloantigen by donor and self antigen (Ag)-presenting cells to host T cells, that differentiate into effector and regulatory cells. Donor dendritic cells (DC), that constitutively express major histocompatibility complex (MHC) and critical T cell costimulatory molecules, are commonly regarded as the principal instigators of rejection, but evidence also exists for DC tolerogenicity, both in the context of allo- and autoimmunity. In experimental animals, liver transplant tolerance is associated with the persistence of donor DC in host lymphoid tissue, in the absence of any form of immunosuppressive therapy (Lu L, Rudert W A, Qian S, et al., Growth of donor-derived dendritic cells from the bone marrow of murine liver allograft recipients in response to granulocyte/macrophage colony-stimulating factor. J Exp Med 1995; 182:379). In human heart and liver transplantation, persistence of donor DC has also been observed in the absence of immunosuppression (O'Connell P J, Burlingham W J. Donor dendritic cell persistence in organ allograft recipients in the absence of immunosuppression, Journal of Leukocyte Biology 1999; 66:301) and in lung recipients, higher levels of donor leukocytes are correlated with better graft survival, and with fewer rejection episodes (McSherry C, Jackson A, Hertz M I, Bolman R M, 3rd, Savik K, Reinsmoen N L. Sequential measurement of peripheral blood allogeneic microchimerism levels and association with pulmonary function. Transplantation 1996; 62:1811; and O'Connell P J, Mba-Jonas A, Leverson G E, et al. Stable lung allograft outcome correlates with the presence of intragraft donor-derived leukocytes. Transplantation 1998; 66:1167). Donor DC that can subvert allogeneic T cell responses in vitro can also prolong the survival of various types of allograft, in some instances indefinitely (Lu L, Rudert W A, Qian S, et al., Growth of donor-derived dendritic cells from the bone marrow of murine liver allograft recipients in response to granulocyte/macrophage colony-stimulating factor. J Exp Med 1995; 182:379; Rastellini C, Lu L, Ricordi C, Starzl T E, Rao A S, Thomson A W. Granulocyte/macrophage colony-stimulating factor-stimulated hepatic dendritic cell progenitors prolong pancreatic islet allograft survival. Transplantation 1995; 60:1366; Fu F, Li Y, Qian S, et al., Costimulatory molecule-deficient dendritic cell progenitors (MHC class II⁺, CD80^{dm}, CD86⁻) prolong cardiac allograft survival in nonimmunosuppressed recipients. Transplantation 1996; 62:659; and Hayamizu K, Huie P, Sibley R K, Strober S. Monocyte-derived dendritic cell precursors facilitate tolerance to heart allografts after total lymphoid irradiation. Transplantation 1998; 66:1285).

The postulated role of donor-derived leukocytes in organ transplant tolerance has generated extensive discussion and debate (Starzl T E, Demetris A J, Murase N, Trucco M, Thomson A W, Rao A S. The lost chord: microchimerism and allograft survival. Immunol Today 1996; 17:577; Bonham CA, Lu L, Thomson A W. Is chimerism necessary for tolerance and how? Curr. Opin. in Organ Transplantation 1997; 2:23; Thomson A W, Lu L. Are dendritic cells the key to liver transplant tolerance? Immunol Today 1999; 20:27; and Wood K, Sachs D H. Chimerism and transplantation tolerance: cause and effect. Immunol Today 1996; 17:584) and provided an impetus for clarification of the possible regulatory function of donor DC in host T cell responses to alloAgs. The concept that interstitial “passenger leukocytes,” in particular DC, are the most important immunogenic component of transplanted whole organs has prevailed for many years (Lechler R I, Batchelor J R. Restoration of immunogenicity to passenger cell-depleted kidney allografts by the addition of donor strain dendritic cells. J Exp Med 1982; 155:31; Talmage D W, Dart G, Radovich J, Lafferty K J. Activation of transplant immunity: effect of donor leukocytes on thyroid allograft rejection. Science 1976; 191:385; Rouabhia M, Germain L, Belanger F, Auger F A. Cultured epithelium allografts: Langerhans cell and Thy-1⁺ dendritic epidermal cell depletion effects on allograft rejection. Transplantation 1993; 56:259; and Larsen C P, Morris P J, Austyn J M. Migration of dendritic leukocytes from cardiac allografts into host spleens. A novel pathway for initiation of rejection. J Exp Med 1990;171:307). Almost a decade ago, Starzl et al. (Starzl T E, Demetris A J, Murase N, Ildstad S, Ricordi C, Trucco M. Cell migration, chimerism, and graft acceptance. Lancet 1992; 339:1579; Starzl T E, Demetris A J, Trucco M, et al. Cell migration and chimerism after whole-organ transplantation: the basis of graft acceptance. Hepatology 1993; 17:1127), used immunohistochemical and molecular biologic techniques to detect donor hematopoietic cells (microchimerism) in blood, and both lymphoid and non-lymphoid tissues of long-surviving, successful human organ allograft recipients. This finding called into question a purely immunogenic role of donor-derived cells, amongst which DC were a consistent, readily detected component. It was proposed that the ability of an organ to be tolerogenic, in the absence or presence of effective immunosuppression, was dependent on its donor leukocyte, and not its parenchymal cell component (Starzl T E et al. (1992)).

The tolerogenic properties of hepatic allografts have long been recognized (reviewed in Qian S, Thai N L, Lu L, Fung J J, Thomson A W. Liver transplant tolerance: mechanistic insights from animal models, with particular reference to the mouse. Transplant Rev 1997; 11:151). In mice, and in certain rat strain combinations, fully MHC-mismatched liver grafts are accepted without immunosuppression, and induce donor-specific tolerance to subsequent heart or skin grafts. Liver allografts are also accepted “spontaneously” between outbred pigs (Calne R Y, Sells R A, Pena J R, et al. Induction of immunological tolerance by porcine liver allografts. Nature 1969; 223:472). In humans, the liver is generally regarded as the least immunogenic of transplanted organs. Its tolerogenic effect seems to result in the long-term acceptance of other organs (e.g., heart or kidney) transplanted simultaneously from the same donor. Co-transplantation of rat heart and liver allografts from the same donor can prevent the development of obliterative arteriopathy in the transplanted heart, a lesion indicative of chronic rejection (Terakura M, Murase N, Demetris A J, Ye Q, Thomson A W, Starzl T E. Lymphoid/nonlymphoid compartmentalization of donor leukocyte chimerism in rat recipients of heart allografts, with or without adjunct bone marrow. Transplantation 1998; 66:350). In a tolerant strain combination, reduction of donor hematopoietic cells in the liver by irradiation before transplantation results in graft rejection (Sun J, McCaughan G W, Gallagher N D, Sheil A G, Bishop G A. Deletion of spontaneous rat liver allograft acceptance by donor irradiation. Transplantation 1995; 60:233). Moreover, the tolerogenic capacity of a leukocyte-depleted liver can be restored after “parking” in a normal donor before transplantation, or after administration of donor leukocytes (Shimizu Y, Goto S, Lord R, et al. Restoration of tolerance to rat hepatic allografts by spleen-derived passenger leukocytes. Transpl Int 1996; 9:593). These observations suggest that, under certain circumstances, donor hematopoietic cells (or a subset thereof) play a role in long-term graft survival/tolerance induction.

The leukocyte lineage(s) that may be involved in the induction of liver tolerance is of crucial relevance in transplant immunology. It has been shown that donor-derived DC persist in, and can be propagated from, the bone marrow (BM) of animals that accept fully-mismatched liver allografts without immunosuppression. This cannot be achieved in animals that acutely reject heart grafts from the same donor strain (Lu L, Rudert WA, Qian S, et al., Growth of donor-derived dendritic cells from the bone marrow of murine liver allograft recipients in response to granulocyte/macrophage colony-stimulating factor. J Exp Med 1995; 182:379), unless both donor BM cells and immunosuppression are administered (Khanna A, Steptoe R J, Antonysamy M A, Li W, Thomson A W. Donor bone marrow potentiates the effect of tacrolimus on nonvascularized heart allograft survival: association with microchimerism and growth of donor dendritic cell progenitors from recipient bone marrow. Transplantation 1998; 65:479). Recent work (Ko S, Deiwick A, Jager M D, et al. The functional relevance of passenger leukocytes and microchimerism for heart allograft acceptance in the rat. Nature Medicine 1999; 5:1292), using a heart transplant model, provided evidence that donor hematopoietic cells were essential for tolerance induction, but the identity of these tolerance-promoting cells was not investigated. Further recent evidence of a critical requirement for donor leukocytes (most likely DC) in organ transplant tolerance has come from observations of the donor-specific blood transfusion (DST) effect (Josien R, Heslan M, Brouard S, Soulillou J P, Cuturi M C. Critical requirement for graft passenger leukocytes in allograft tolerance induced by donor blood transfusion. Blood 1998; 92:4539). Thus, injection of donor-type, but not third party DC, restored tolerance to passenger leukocyte-depleted heart allografts in DST-treated rats.

Additional recent studies revealed for the first time, that immature ‘donor DC could induce alloAg-specific T cell hyporesponsiveness in vitro. More significantly, recent work has shown that such immature DC, including liver-derived DC, can prolong allograft (including skin graft) survival; in some instances; indefinite, donor-specific graft survival is induced (Rastellini C, Lu L, Ricordi C, Starzl T E, Rao A S, Thomson A W. Granulocyte/macrophage colony-stimulating factor-stimulated hepatic dendritic cell progenitors prolong pancreatic islet allograft survival. Transplantation 1995; 60:1366; Fu F et al., (996); Hayamizu K, Huie P, Sibley R K, Strober S. Monocyte-derived dendritic cell precursors facilitate tolerance to heart allografts after total lymphoid irradiation. Transplantation 1998; 66:12855-7; Lu L, McCaslin D, Starzl T E, Thomson A W. Bone marrow-derived dendritic cell progenitors (NLDC 145⁺, MHC class II⁺, B7-1^{dm}, B7-2⁻) induce alloantigen-specific hyporesponsiveness in murine T lymphocytes. Transplantation 1995; 60:1539; Lutz MB, Suri R M, Niimi M, et al. Immature dendritic cells generated with low doses of GMCSF in the absence of Interleukin 4 (IL-4) are maturation resistant and prolong allograft survival in vivo. Eur J Immunol 200; 30:1813; Hayamizu K, Zeng D, Huie P, et al. Donor blood monocytes but not T or B cells facilitate long-term allograft survival after total lymphoid irradiation. Transplantation 1998; 66:585; and Gozzo J, Mash S, DeFazio S. Extension of graft survival with pulsed administration of donor dendritic cells. Transplant Proc 1998; 31:1196). In addition, there is evidence that persistence of donor liver-derived DC may be important in long-term maintenance of transplantation tolerance; and in the prevention of chronic rejection in challenge heart allografts (Demetris A J, Murase N, Ye Q, et al. Analysis of chronic rejection and obliterative arteriopathy. Possible contributions of donor antigen-presenting cells and lymphatic disruption. Am J Pathol 1997; 150:563). Collectively, these findings suggest that donor-derived DC have potential to subvert recipient T cell responses to alloAg, by as yet ill-defined mechanisms.

Published data suggest that DC have capacity to regulate immune reactivity by a variety of mechanisms (Thomson A W, Lu L. Are dendritic cells the key to liver transplant tolerance? Immunol Today 1999; 20:27; Steptoe R J, Thomson A W. Dendritic cells and tolerance induction. Clin Exp Immunol 1996; 105:397; Morelli A E, Thomson A W. Dendritic cells as regulators of tolerance and immunity: relevance to transplantation. Graft 1999; 28:34; Lotze M T, Thomson A W. Dendritic cells. Biology and Clinical Applications. San Diego: Academic Press, 1999:1-733; Lu L, Khoury S J, Sayegh M H, Thomson A W. Dendritic cell tolerogenicity and prospects for cell-based therapy of allograft rejection and autoimmunity: In: Lotze M T, Thomson A W, eds. Dendritic cells. Biology and Clinical Applications. San Diego: Academic Press, 1999:487; Banchereau J, Steimnan R M. Dendritic cells and the control of immunity. Nature 1998; 392:245). These mechanisms may not be mutually exclusive. It has been reported that DC whose allostimulatory function is impaired either by incomplete maturation, selective blockade of costimulatory molecules, the influence of specific cytokines (e.g., IL-10 or transforming growth factor (TGF) β), or genetic engineering, can induce alloAg-specific T cell hyporesponsiveness (anergy) or apoptosis in vitro, and suppress immune reactivity (reviewed in refs Steptoe R J et al. (1996); Morelli A E et al. (1,999); Lu L, et al. (1999); and Thomson A W, Lu L. Dendritic cells as regulators of immune reactivity: implications for transplantation. Transplantation 1999; 68:1).

Expression of molecules associated with the induction of apoptosis (for example, FasL [CD95L] (Suss G, Shortman K. A subclass of dendritic cells kills CD4 T cells via Fas/Fas-ligand-induced apoptosis. J Exp Med 1996; 183:1789 and Lu L, Qian S, Hershberger P A, Rudert W A, Lynch D H, Thomson A W. Fas ligand (CD95L) and B7 expression on dendritic cells provide counter-regulatory signals for T cell survival and proliferation. J Immunol 1997; 158:5676) or nitric oxide (NO) (Lu L, Bonham C A, Chambers F G, et al. Induction of nitric oxide synthase in mouse dendritic cells by interferon (IFN) γ (IFN-γ), endotoxin, and interaction with allogeneic T cells: nitric oxide production is associated with dendritic cell apoptosis. J Immunol 1996; 157:3577 and Fehsel K, Kroncke K D, Meyer K L, Huber H, Wahn V, Kolb-Bachofen V. Nitric oxide induces apoptosis in mouse thymocytes. J Immunol 1995; 155:2858) may render DC capable of subverting T cell responses by promoting activation-induced cell death. Blockade of the B7/CD28 pathway by cytotoxic T lymphocyte Antigen 4 (CTLA4) (CD152) immunoglobulin (Ig) significantly increases DC-induced apoptosis of alloactivated T cells (Lu L. et al. (1997)) that appears to be mediated, at least in part, via the Fas pathway. Death-inducing ligands on DC other than FasL, such as tumor necrosis factor (TNF) receptor apoptosis-inducing ligand (TRAIL) are being investigated. There is recent evidence that these molecules may be differentially expressed on human DC subsets (Fanger N A, Maliszewski C R, Schooley K, Griffith T S. Human dendritic cells mediate cellular apoptosis via tumor necrosis factor-related apoptosis-inducing ligand (TRAIL). J Exp Med 1999; 190:1155).

Immune deviation, that is, skewing the T helper 1 (Th1)/T helper 1 (Th2) cell balance to Th2 cells, has received considerable attention as a mechanism that may underlie tolerance induction. Several groups have shown that DC can induce immune deviation. Thus, Ag-specific suppression of cell-mediated immunity achieved by intravenous (i.v.) administration of Ag-pulsed Langerhans cells or splenic DC, is likely achieved via selective activation of Th2 cells (Morikawa Y, Tohya K, Ishida H, Matsuura N, Kakudo K. Different migration patterns of antigen presenting cells correlate with Th1/Th2-type responses in mice. Immunology 1995; 85:575). DC grown in prostaglandin (PG) E₂ are unable to secrete IL-12, so that Th2 cell development is promoted (Kalinski P, Hilkens C M, Snijders A, Snijdewint F G, Kapsenberg M L. IL-12-deficient dendritic cells, generated in the presence of prostaglandin E2, promote type 2 cytokine production in maturing human naive T helper cells. J Immunol 1997; 159:28). IL-10 skews the Th1/Th2 balance to Th2 cells, by blocking IL-12 synthesis by DC (De Smedt T, Van Mechelen M, De Becker G, Urbain J, Leo O, Moser M. Effect of interleukin-10 on dendritic cell maturation and function. Eur J Immunol 1997; 27:1229). In transplantation, prolongation of skin graft survival by portal venous immunization with donor DC is associated with Th2 polarization (Gorczynski R M, Cohen Z, Fu X M, Hua Z, Sun Y, Chen Z. Interleukin-l 3, in combination with anti-interleukin-12, increases graft prolongation after portal venous immunization with cultured allogeneic bone marrow-derived dendritic cells. Transplantation 1996; 62:1592). In autoimmune disease, a protective effect of autoAg-pulsed DC in experimental allergic encephalomyclitis appears to be mediated by immune deviation (Khoury S J, Sayegh M H, Hancock W W, Gallon L, Carpenter C B, Weiner H L. Acquired tolerance to experimental autoimmune encephalomyelitis by intrathymic injection of myelin basic protein or its major encephalitogenic peptide. J Exp Med 1993; 178:559; and Khoury S J, Gallon L, Verburg R R, et al. Ex vivo treatment of antigen-presenting cells with CTLA4Ig and encephalitogenic peptide prevents experimental autoimmune encephalomyelitis in the Lewis rat. J Immunol 1996; 157:3700).

Dendritic cells can be generated from CD34⁺ hematopoietic progenitors via several pathways (Shortman K, Caux C. Dendritic cell development: multiple pathways to nature's adjuvants. Stem Cells 1997; 15:409). The hematopoietic growth factors, c-kit ligand and Fms-like tyrosine kinase 3 ligand (Flt3L) promote growth of DC progenitors. Granulocyte-macrophage colony-stimulating factor (GM-CSF) and IL-3 enhance DC differentiation, while TNF and CD40L promote myeloid (M)DC maturation. Monocytes differentiate into MDC in response to GM-CSF and IL-4. In contrast to MDC, lymphoid (L)DC, identified initially as constitutive DC within the thymus, share a common progenitor with T cells (Wu L, Li CL, Shortman K. Thymic dendritic cell precursors: relationship to the T lymphocyte lineage and phenotype of the dendritic cell progeny. J Exp Med 1996; 184:903).

The LDC of mouse thymus delete developing T cells with autoreactive potential (Brocker T, Riedinger M, Karjalainen K. Targeted expression of major histocompatibility complex (MHC) class II molecules demonstrates that dendritic cells can induce negative but not positive selection of thymocytes in vivo. J Exp Med 1997; 185:541 and Steinman R M, Pack M, Inaba K. Dendritic cells in the T-cell areas of lymphoid organs. Immunol Rev 1997; 156:25). It has also been suggested that LDC are involved in the maintenance of peripheral tolerance (Shortman K. et al. (1997); Steinman R M et al. (1997); and Inaba K, Pack M, Inaba M, Sakuta H, Isdell F, Steinman R M. High levels of a major histocompatibility complex II-self peptide complex on dendritic cells from the T cell areas of lymph nodes. J Exp Med 1997; 186:665). In mice, LDC can induce T cell proliferation without concomitant cytokine (IL-2, IL-3, IFN-γ and granulocyte macrophage-colony stimulating factor (GM-CSF)) production (Shortman K. et al. (1997) and Kronin V, Winkel K, Suss G, et al. A subclass of dendritic cells regulates the response of naive CD8 T cells by limiting their IL-2 production. J Immunol 1996; 157:3819). They kill CD4+T cells via Fas (CD95)-mediated apoptosis (Suss G et al. (1996)). In humans, so-called “plasmacytoid T cells” that express CD4 and MHC class II develop into DC after stimulation with IL-3 and CD40L, and are therefore regarded as putative precursors of plasmacytoid DC (DC2, see below) (Grouard G, Rissoan M C, Filgueira L, Durand I, Banchereau J, Liu Y J. The enigmatic plasmacytoid T cells develop into dendritic cells with interleukin (IL)-3 and CD40-ligand. J Exp Med 1997; 185:1101). Human MDC, that induce Th1 cell differentiation, and human plasmacytoid DC, that induce Th2 cells, are referred to as DC1 and DC2 respectively (Rissoan M C, Soumelis V, Kadowaki N, et al. Reciprocal control of T helper cell and dendritic cell differentiation. Science 1999; 283:1183 and Siegal F P, Kadowaki N, Shodell M, et al. The nature of the principal type 1 interferon-producing cells in human blood. Science 1999; 284:1835). Due to their in vitro functional properties, it has been suggested (Suss G et al. (1996); and Fazekas de St Groth B. The evolution of self-tolerance: a new cell arises to meet the challenge of self-reactivity. Immunol Today 1998; 19:448) that LDC may be DC specialized for tolerance induction. Significantly, it has been observed that murine LDC of donor origin infused systemically one week before organ transplant, can markedly prolong vascularized cardiac allograft survival (O'Connell P J, Li W, Wang Z, Specht S M, Logar A J, Thomson A W. Immature and mature CD8α+ dendritic cells prolong the survival of vascularized heart allografts. J Immunol 2002 Jan 1;168(1):143-54). It is unknown, however, if the presence or predominance of the putatively tolerogenic plasmacytoid DC subset, that can be propagated from blood (Grouard G, et al. (1997)), correlates with the quiescent clinical state, and with hypo/unresponsiveness to donor alloAgs.

### SUMMARY

It now has been found that elevated pDC2/pDC1 in peripheral blood of graft recipients, particularly allograft liver recipients, correlates with tolerance of the graft in the graft recipient. Provided, therefore, is a method of identifying tolerance in a graft recipient, including the step of quantitating the number of pDC1 cells and the number of pDC2 cells in a peripheral blood sample of the recipient. By calculating the pDC2/pDC1 for the sample graft tolerance can be predicted. Quantitating the numbers of pDC2 and pDC1 cells in a sample may be performed by flow cytometry. pDC1 and pDC2 populations may be identified by cellular markers, for example, pDC1 cells may be characterized, counted and/or sorted by the phenotype HLA-DR⁺lin⁻CD11c⁺CD123^{lo} and pDC2 cells may be characterized, counted and/or sorted by the phenotype HLA-DR⁺lin⁻CD11c⁻CD¹²³⁺, where lin markers are CD3, CD14, CD19, CD20. By flow cytometry analysis according to those phenotypes, a typical graft recipient having a pDC2/pDC1 of greater than from about 0.20 to about 0.30 is likely to be tolerant of the graft. The pDC1/pDC2 either can be determined from direct analysis of cells in a peripheral blood sample, or DC1 and DC2 cells can be cultured from a peripheral blood sample and the numbers of pDC1 and pDC2 cells in the peripheral blood sample can be estimated from the growth of the DC1 and DC2 cells in culture. Other methods of quantitating pDC2/pDC1 may be used to provide an equivalent threshold ratio above which graft tolerance would be likely.

A method also is provided for identifying tolerance in a graft recipient that includes the steps of staining a sample of peripheral blood mononuclear cells (PBMC) of the graft recipient with one or more binding reagents for differentiating pDC1 cells from pDC2 cells and determining the number of pDC1 cells and the number of pDC2 cells in the sample. As above, if the pDC2/pDC1 is high, tolerance can be predicted.

A set of binding reagents also is provided for use in identifying graft tolerance in a graft recipient. The set includes suitable binding reagents, typically fluorochrome-conjugated monoclonal antibodies, permitting quantitation of pDC1 cells and pDC2 cells in the peripheral blood of a graft recipient.

### BRIEF DESCRIPTION OF THE DRAWINGS

FIGS. 1A-C are a graphs presenting the data provided in Table 1.

FIGS. 2A-F are bivariate plots illustrating flow cytometric analysis of PBMC used in generating the data presented in Table 1.

### DETAILED DESCRIPTION

A method is provided for determining the likelihood that a graft recipient will tolerate the graft. To this end, it has been found that a higher ratio of pDC2 cells to pDC1 cells in the patient correlates with tolerance. For consistency, the following definitions apply.

“pDC1” refers to monocytoid DC found in peripheral blood. Monocytoid dendritic cells are considered to be the equivalent of murine myeloid DC. “pDC2” refers to plasmacytoid DC found in peripheral blood, which are equivalent to murine plasmacytoid DC. pDC1 and pDC2 terminally differentiate into DC1 and DC2 cells, respectively.

“Tolerant” and “tolerance” refers to the case where a non-self graft does not induce an immune response that leads to donor tissue rejection, even without immunosuppressant therapy.

The term “graft” refers generally to any tissue or cell transplant or transfer, and includes both allografts (transplants between different members of the same species) and xenografts (trans-species transplants). The term “graft” also includes autografts (of self origin) and isografts (transplantation between genetically identical donors and recipients, such as between monozygotic twins), but rejection in those cases is not common.

As used herein, the term “binding reagent” and like forms refer to any compound, composition or molecule capable of specifically or substantially specifically (that is with limited cross-reactivity) binding another compound or molecule. Typically, the binding reagents are antibodies, preferably monoclonal antibodies, or derivatives or analogs thereof, including without limitation: Fv fragments; single chain Fv (scFv) fragments; Fab′ fragments; F(ab′)2 fragments; camelized antibodies and antibody fragments; and multivalent versions of the foregoing. Multivalent binding reagents also may be used, as appropriate, including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments. Other binding reagents include, without limitation, aptamers.

As shown herein, the pDC2/pDC1 ratio correlates with graft tolerance, particularly allograft tolerance. The methods described in the examples below differentiate pDC1 from pDC2 by flow cytometry according to the following method. First, PBMC are obtained and are stained with four specificities of antibodies, each conjugated with a fluorochrome having a different emission spectrum, as follows: eye-conjugated anti-HLA-DR; FITC-conjugated anti-CD3, anti-CD14, anti-CD19 and anti-CD20 (lineage or “lin” markers); R-phycoerythrin-conjugated anti-CD123; and APC-conjugated anti-CD11c (Cyc=Cy-Chrome™ (BD Biosciences of San Jose, Calif.), FITC=fluorescein isothiocyanate, PE=R-phycoerythrin and APC=allophycocyanin). As used herein, when a fluorochrome is attached or conjugated to a binding reagent or antibody, it is covalently linked to that binding reagent or antibody in a manner retaining specificity of the binding reagent or antibody and retaining fluorescent properties of the fluorochrome, permitting detection of the fluorochrome both when attached to the binding reagent and when the binding reagent-fluorochrome conjugate is bound to, or otherwise associated with a cell.

The stained PBMC are then characterized (or sorted) by flow cytometry. Lymphocytes are first quantified (or selected) by forward and side scattering. As used herein, all percentages are in reference to the number of gated lymphocytes. Cells positive for HLA-DR, but lin negative were then gated and divided into two populations: CD123^{lo} CD11c⁺(pDC1) and CD123^{+(or h1)} CD11c⁻ (pDC2). By this selection method, and in view of the data presented herein, patients exhibiting a pDC2/pDC1 (number of pDC2 cells÷number of pDC1 cells) of greater than from about 0.20 to about 0.30 were more likely to tolerate a graft than patients having a pDC2/pDC1 lower than from about 0.20 to about 0.30. The ratios stated above are prefaced by the word “about,” which reflects the patient-to-patient variance that will be present, and even variance for any given patient over time.

The above-described assay is one way to determine pDC2/pDC1 in PBMC, and may be a preferred method in many instances. However, any method for measuring the pDC2/pDC1 ratio in peripheral blood may be used to determine a graft recipient's state of immune tolerance. In one alternative method, as shown in Example 2, below, the pDC1/pDC2 also can be determined by culturing DC1 and DC2 cells from a peripheral blood sample and the numbers of pDC1 and pDC2 cells in the peripheral blood sample can be estimated from the growth (for example growth curves) of the DC1 and DC2 cells in culture.

Further, as is typical in the field of the present invention, especially with respect to newly discovered cell types such as pDC2 cells, additional lineage markers may be discovered, and better or different assay methods might be determined that would provide other methods for determining pDC2/pDC1. For this reason, as used herein, this ratio may be referred to as being equivalent to a pDC2/pDC1 obtained by flow cytometry using [HLA-DR⁺lin⁻ (CD3⁻, CD14⁻, CD19⁻ and CD20⁻) CD123⁺ CD11c⁻] and [HLA-DR⁺lin⁻ CD123^{lo} CD11c⁺] for pDC2 and pDC1 populations, respectively. A “high pDC2/pDC1” refers to a pDC2/pDC1 at which it is more likely, in a population of allograft recipients, that any given patient will tolerate an allograft rather than reject the allograft. In other words, a “high pDC2/pDC1” is a pDC2/pDC1 that is significantly higher (p<0.05) than a pDC2/pDC1 for patients who would reject an allograft upon removal of immunosuppressive therapy. A pDC2/pDC1 value “predictive of tolerance” is a pDC2/pDC1 value sufficiently high to provide an indication that a patient might safely discontinue immunosuppressant therapy.

For the flow cytometry method described herein, the antibodies used to identify the HLA-DR, lin, CD123 and CD11c markers can be any antibody capable of identifying these markers, and are not limited to the specific monoclonal antibody clones listed herein. The choice of antibody is a matter of experimental design, convenience and/or personal preference. Similarly, the choice of fluorochromes to conjugate to the antibodies and the cell detection, counting and sorting method(s) are a matter of experimental design, convenience and/or personal preference. As discussed above, a “binding reagent” may be equally suited for use in flow cytometry analysis as an antibody.

In any case, the binding reagents used to differentiate the pDC1 and pDC2 cells are attached to, or inherently contain, a tag that renders pDC1 cells and pDC2 cells detectably different. In the examples below, the tag is a fluorochrome, which may be attached to or incorporated within a bead, as is known in the art. Another “tag” is an affinity bead, such as, without limitation, a magnetic-bead, that permits mechanical separation of the cell types. When the tag is a fluorochrome, virtually any fluorochrome and/or combinations thereof combination may be used to differentiate cell types. In the examples below, four different fluorochromes are used to facilitate quantitation of different cell types by flow cytometry. In multivariate flow cytometry, as described in the examples below, so long as the fluorochromes attached to any class of binding reagent is detectably different, it is suitable for such a use. By “detectably different” it is meant that two different binding reagent-tag combinations, when bound, or otherwise associated with a cell, or cells, can be differentially detected using any selected detection and quantitation technology. For example, and without limitation, when the detection and quantitation technology is fluorescent flow cytometry, each binding reagent to be differentially detected would have a different excitation and/or emission spectrum when bound, or otherwise associated with a cell. Examples of suitable fluorochromes include, without limitation, cyanine dyes, dipyrromethene boron difluoride dyes, FITC, and stokes-shifting dye conjugates, such as, without limitation, Cy-Chrome™ (a tandem fluorochrome composed of R-phycoerythrin (PE), which is excited by 488 nm light and serves as an energy donor, coupled to the cyanine dye Cy5™, which acts as an energy acceptor and fluoresces at 670 nm) which is used in the Examples below, and fluorochromes described in U.S. Pat. Nos. 6,008,373 and 6,130,094.

When a binding reagent is “bound or otherwise associated” with a cell, it is meant that the binding or association is specific for the target (epitope when referring to antibodies) of the binding reagent.

Provided herein also is a “set” of binding reagents, referring to two or more different binding reagents, such as, without limitation anti-HLA-DR, anti-lin, anti-CD123 and anti-CD11c antibodies, provided in a single container, or in any combination and quantity in separate containers for use in differentially detecting, quantitating or sorting pDC1 and PDC2 cells. The “set” is a panel of binding reagents capable of distinguishing pDC1 and pDC2 cells in the spirit of the present disclosure. Binding reagents of the “set” typically are antibodies. In one embodiment, the antibodies are conjugated with appropriate fluorochromes, as described herein, to permit multivariate flow cytometry to identify, quantitate and/or sort pDC1 and pDC2 cells. The set of binding reagents can be provided as part of a kit, which may be designed to facilitate quantitation and/or sorting of pDC1 and pDC2 cells. The kit may contain additional reagents, such as, without limitation, buffers, blocking reagents (for instance, goat serum) and empty vials or multi-well plates. The binding reagents, as well as other ingredients, may be provided in a single- or multi-compartment cartridge for use in an automated sample processing system. The binding reagents and other ingredients can be provided in any useful form, including, without limitation, in aqueous, dry or lyophilized form.

### EXAMPLE 1

Rare event flow cytometric analysis was used to examine circulating DC subsets in stable liver transplant patients off all immunosuppression, in a prospective drug withdrawal group undergoing progressive weaning, in patients with a history of rejection in which weaning had never been attempted, and in normal healthy controls. These data reveal higher relative incidences of pDC2 relative to pDC1 in prospective weaning and tolerant patients compared with subjects with a history of rejection.

Study population—Twenty clinically stable liver transplant patients with normal graft function were included. The mean age at transplantation was 9.9 years (range 0.4-48 years). Diagnoses at liver transplantation included biliary artresia (n=6), metabolic disease (n=4), autoimmune liver disease (n=5), cholestatic syndromes (n=3), Wilson's Disease (n=1) and cryptogenic cirrhosis (n=1). Patients off immunosuppression have been free from immunosuppression for a mean of 2.7 years with a range of 0.3-7.4 years and were weaned off drugs by physician direct protocol (n=2) or emergently for life-threatening infectious disease indications (n=4). Also included were patients who were treated with minimal immunosuppressants and were candidates for weaning from immunosuppressants therapy (“prospective weaning group”) and patients who were either weaned or were completely removed from immunosuppressants therapy, but required reinstitution of immunosuppressant therapy. The normal control population consisted of 10 adults with a mean current age of 34 years.

Isolation of PBMC—Peripheral venous blood samples from liver transplant patients and healthy controls were collected in heparinized tubes. The blood samples were rocked slowly, overnight (18 hr) at room temperature. Peripheral blood mononuclear cells (PBMC) were then isolated by Ficoll-Paque (Amersham Pharmacia Biotech AB, Uppsala, Sweden) density gradient centrifugation. Briefly, the anticoagulant-treated peripheral blood was dispensed in into 50 ml polypropylene conical tubes (PP tube) and was diluted with 1× PBS/2 mM EDTA. The blood was underlayed with Ficoll-Paque Plus (ficoll, 1.077 g/ml) and centrifuge at room temperature for 40 min at 400 g. The cells were washed twice with 1× PBS/2 mM EDTA and suspended in cell sorting buffer, defined below. PBMC were counted and their viability determined by Trypan Blue exclusion.

Immunofluorescent Cell Staining an Flow Cytometric Analysis—PBMC were suspended at a concentration of 2.5×10⁶/ml in cell staining buffer (CSB) (phosphate-buffered saline (PBS) supplemented with 1% fetal calf serum (FCS) and 0.1% sodium azide). Cells were incubated in normal goat serum (Gibco-BRL, cat# 16210-064) to block non-specific binding of antibodies to Fc receptors. The cells (0.2 to 0.5×10⁶) were aliquoted into polystyrene tubes and washed ×1 with CSB. Cells were stained for 30 min on ice with FITC-conjugated anti-CD3 (clone SP34), CD14 (clone M5E2), CD19 (clone J4.119), CD20 (clone 2H7); Cy-Chrome™-conjugated anti-HLA-DR (clone G46-6); PE-conjugated anti-CD123 (IL-3Rα, clone 7G3); APC-conjugated anti-CD 11c (clone S-HCL-3). All antibodies except anti-CD19 (Beckman Coulter) were purchased from, and are commercially available from BD Biosciences). Stained cells were washed ×1 in CSB, suspended in PBS and fixed by adding 45 paraformaldehyde to a 1% final concentration. Four-color flow cytometric analysis of 50,000-250,000 cells was performed using a Coulter Epics Elite ESP (Beckman Coulter) in conjunction with EXPO32™ software (Applied Cytometry Systems of Sacramento, Calif.). Cells that were negative for CD3, CD 14, CD19 and CD20 (lineage) and positive for HLA-DR were gated and analyzed for CD 11c and CD 123 expression. The precursors (p) of monocytoid DC (pCD1) and plasmacytoid DC (pDC2) were identified as HLA-DR⁺lin⁻CD11c⁺CD123^{lo} (pDC1) and HLA-DR⁺lin⁻CD11c⁻CD123⁺ (pDC2).

Results—PBMC were isolated from patients successfully withdrawn from immunosuppression following liver transplantation (Group A), those undergoing prospective drug weaning (Group B), those in whom drug withdrawal failed or has not been attempted (Group C) and in normal controls. Total DC were identified as HLA-DR⁺ and lineage marker (CD3, CD14, CD 19, CD20) negative cells on four-color cytometric analysis. Subpopulations of HLA-DR⁺lin⁻CD11c⁺CD123^{lo} (pDC1) and HLA-DR⁺lin⁻CD11c⁻CD123⁺ (pDC2) cells were further quantified.

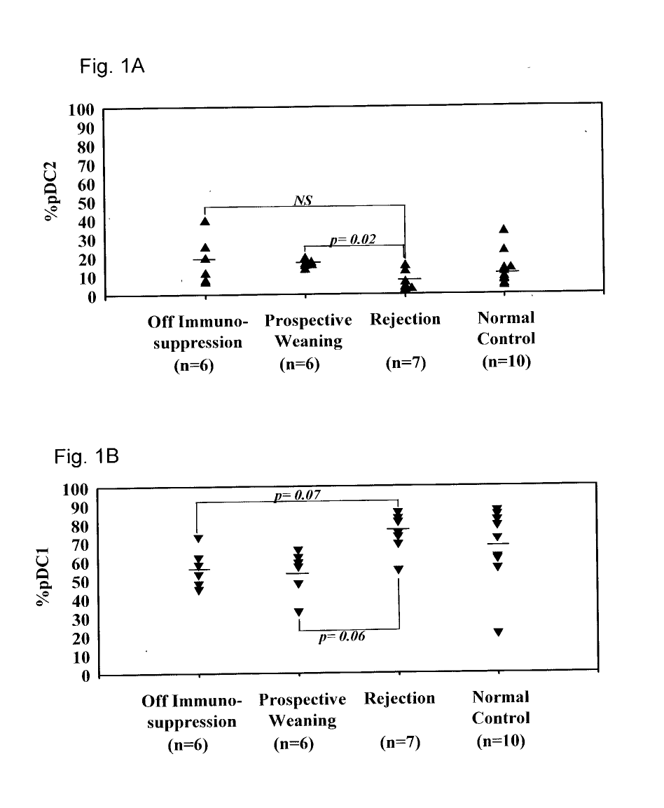

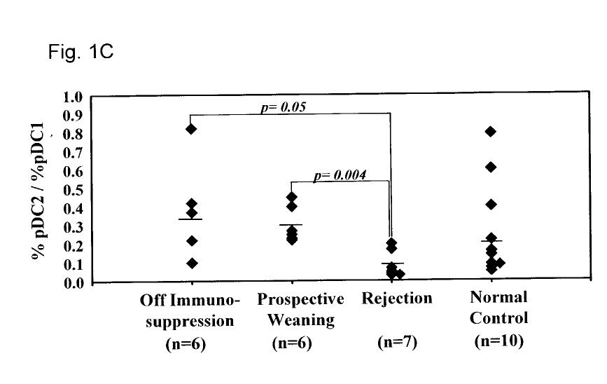

The incidences of DC subsets for subjects in each study group are shown in Table 1, below. These data are presented graphically in FIGS. 1A-C. The pDC2/pDC1 (%pDC2/%pDC1) also is presented. The pDC2 population was increased significantly in the prospective weaning population over the rejection group, and with respect to the pDC1 subsets. When the data from pDC1 and pDC2 subsets were combined as a ratio for each patient, patients who were off immunosuppression had a higher pDC2/pDC1 than those who rejected (p=0.05). Patients undergoing prospective weaning also had a significantly higher ratio than the patients who had a history of rejection, or had experienced rejection during a failed attempt at weaning (p=0.004). The patients off immunosuppression and those prospectively weaning were in fact similar to normal controls.

TABLE 1

| | | % | % | % | % pDC2/ | |
|---|---|---|---|---|---|---|
| Group | n = | pDC | pDC2 | pDC1 | % pDC1 | Statistic |
| Successful | 6 | 2.3 | 17.9 | 56.4 | 0.34 | Mean |
| Weaning | | 1.2 | 12.8 | 10.2 | 0.27 | Std. Dev. |
| (tolerant, off | | 1.1 | 6.3 | 44.9 | 0.10 | Minimum |
| immunosuppression) | | 3.9 | 39.5 | 72.9 | 0.82 | Maximum |
| Prospective | 6 | 1.3 | 15.6 | 54.3 | 0.30 | Mean |
| Weaning | | 0.6 | 2.0 | 12.1 | 0.10 | Std. Dev. |
| | | 0.8 | 13.1 | 33.0 | 0.22 | Minimum |
| | | 2.4 | 19.1 | 66.4 | 0.45 | Maximum |
| Rejection | 7 | 2.7 | 6.4 | 74.5 | 0.09 | Mean |
| | | 1.0 | 5.0 | 11.0 | 0.07 | Std. Dev. |
| | | 1.0 | 2.2 | 54.6 | 0.03 | Minimum |
| | | 3.8 | 14.8 | 85.8 | 0.20 | Maximum |
| Normal | 10 | 2.2 | 9.9 | 67.8 | 0.21 | Mean |
| Control | | 1.1 | 3.5 | 21.3 | 0.22 | Std. Dev. |
| | | 1.1 | 5.1 | 20.4 | 0.01 | Minimum |
| | | 4.3 | 14.6 | 83.9 | 0.72 | Maximum |

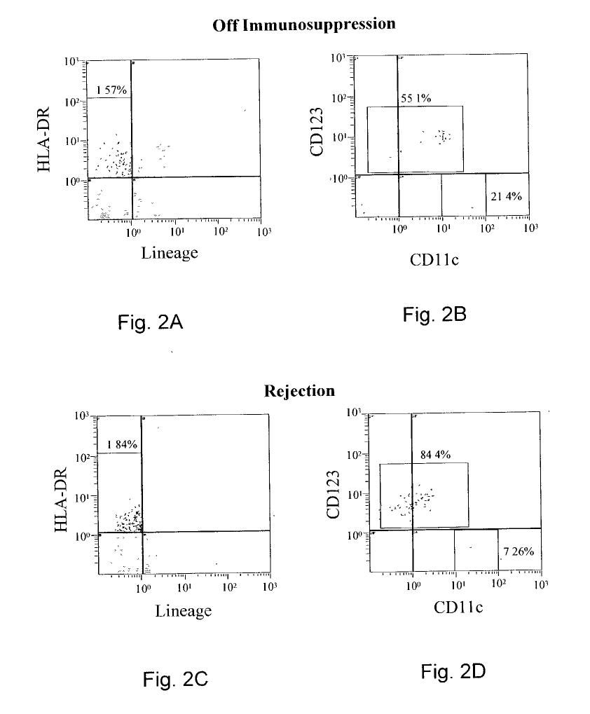

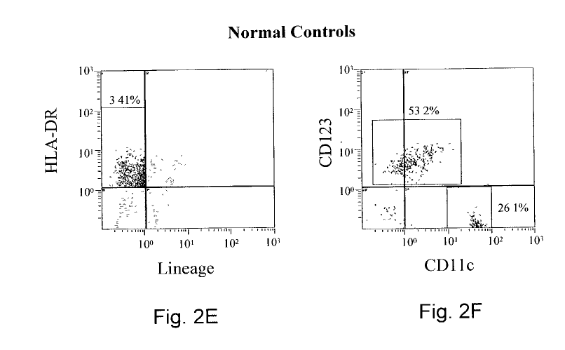

FIGS. 2A-F is provided to illustrate the general flow cytometry gating strategy. Data from three cases are provided, including: a patient off immunosuppression, a rejection patient and a normal control. The bivariate plots shown in panels A, C and E show HLA-DR (x-axis) versus lineage (y-axis). The bivariate plots shown in panels B, D and F show CD11c (x-axis) versus CD123 (y-axis). Panels B and D illustrate the significant differences in pDC2/pDC1 for a patient off immunosuppression (21.4%/55.1% =0.39) as compared to a rejection patient (7.26%/84.4% =0.09).

Conclusion—These data suggest that pDC2/pDC1 is significantly higher in patients successfully withdrawn from immunosuppression as well as in those on low levels of immunosuppression undergoing prospective drug weaning as compared to those patients requiring ongoing maintenance immunosuppression. Further studies are ongoing in a larger patient population.

### EXAMPLE 2

To generate DC1, blood monocytes (specify), isolated by standard methods, are cultured with GM-CSF and IL-4 for 5 days in RPMI-1640 with 10% FCS; the resulting immature DC1 can then be matured by exposure for 24 hr to CD40L or anti-CD40 mAb (Rissoan MC et al. (1999)). To generate DC2, CD4⁺ CD11c⁺ lin⁻plasmacytoid cells are isolated (>98% purity) from peripheral blood following immunomagnetic bead depletion of CD3⁺, CD 14⁺, CD 19⁺, CD20⁺ and, optionally, CD56⁺ cells, and sorting (Grouard G et al. (1997)), then cultured with rh IL-3 (±CD40 ligation) for five to seven days. Growth curves for both DC1 and DC2 cells grown in culture would be indicative of initial ratios of pDC1 and pDC2 cells in peripheral blood.

### EXAMPLE 3

As shown above, pDC2 and pDC1 cells can be distinguished by flow sorting. These sorted cells may be subjected to PCR analysis for the sex-determining region of the Y chromosome (where the donor is male and the recipient female) or for mismatched donor (HLA) alleles—both by standard methods. It has previously been shown using immunocytochemical and molecular biologic techniques, that DC of donor origin (donor MHC class I/II⁺) can be identified in cell populations expanded from the BM or blood of liver allograft recipients (Lu L et al. (1995); Thomson A W, Lu L, Wan Y, Qian S, Larsen C P, Starzl T E. Identification of donor-derived dendritic cell progenitors in bone marrow of spontaneously tolerant liver allograft recipients. Transplantation 1995; 60:1555; Rugeles M T, Aitouche A, Zeevi A, et al. Evidence for the presence of multilineage chimerism and progenitors of donor DC in the peripheral blood of bone marrow-augmented organ transplant recipients. Transplantation 1997; 64:735). Quantitative and multiplexed PCR methods (for instance TAQMAN and similar methods), which are broadly known, may be employed, as appropriate, to quantitate relative numbers of donor and host DC. For instance, when host-donor differentiation is based on sex, PCR amplification of a target sequence in the sex-determining region of the Y chromosome may be conducted in a multiplexed, quantitative PCR reaction relative to a control PCR amplification common to both host and donor.

The above invention has been described with reference to the preferred embodiment. Obvious modifications and alterations will occur to others upon reading and understanding the preceding detailed description and the claims. It is intended that the invention be construed as including all such modifications and alterations.

I claim:

1. A method of identifying tolerance in a graft recipient, comprising the step of quantitating the number of pDC1 cells and the number of pDC2 cells in a peripheral blood sample of the recipient.

2. The method of claim 1, wherein the quantitating step is performed by flow cytometry.

3. The method of claim 1, wherein the quantitation step includes the steps of:
(a) identifying a population of peripheral blood mononuclear cells in the sample; and
(b) quantitating HLA-DR⁺lin⁻CD11c⁺CD123^{lo} pDC1 cells and HLA-DR⁺lin⁻CD11c⁻CD123⁺ pDC2 cells in the population of peripheral blood mononuclear cells.

4. The method of claim 3, wherein the step of identifying a population of peripheral blood mononuclear cells is performed by light scattering.

5. The method of claim 1, further comprising the step of determining if a ratio of the number of pDC2 cells to the number of pDC1 cells is greater than a value predictive of tolerance in the recipient.

6. The method of claim 5, wherein the value predictive of tolerance in the recipient is a high pDC2/pDC1.

7. The method of claim 5, wherein the value predictive of tolerance in the recipient is a pDC2/pDC1 equivalent to a pDC2/pDC1 of at least about 0.20, obtained by flow cytometry using HLA-DR⁺lin⁻CD123⁺CD11c⁻ to identify pDC2 cells and HLA-DR⁺lin⁻CD123^{lo}CD11c⁺, wherein lin⁻ is CD3⁻CD14⁻CD19⁻CD20⁻.

8. The method of claim 1, wherein the graft is a liver allograft.

9. The method of claim 1, further comprising the steps of:
(a) culturing DC1 cells from cells of the peripheral blood sample;
(b) culturing DC2 cells from cells of the peripheral blood sample; and
(c) estimating the pDC2/pDC1 in the peripheral blood sample from the growth of the DC1 and DC2 cells.

10. A method of identifying tolerance in a graft recipient, comprising the steps of;
(a) staining a sample of peripheral blood mononuclear cells of the graft recipient with one or more binding reagents for differentiating pDC1 cells from pDC2 cells; and
(b) determining the number of pDC1 cells and the number of pDC2 cells in the sample.

11. The method of claim 10, wherein the binding reagents are antibodies.

12. The method of claim 10, wherein two or more different binding reagents are used to stain the cells and at least two of the different binding reagents are detectably different from each other when bound or otherwise associated to cells.

13. The method of claim 12, wherein the detectably different binding reagents are fluorochrome-conjugated antibodies.

14. The method of claim 10, wherein the binding reagents include an anti-CD123 antibody attached to a first fluorochrome and an anti-CD11c antibody attached to a second fluorochrome, each of the first and second fluorochromes having one or both of a detectably different excitation and emission spectrum.

15. The method of claim 14, the binding reagents further comprising an anti-HLA-DR attached to a third fluorochrome and anti-lineage antibodies, consisting of anti-CD3, anti-CD14, anti-CD19 and anti-CD20 antibodies, attached to a fourth fluorochrome, each of the first, second, third and fourth fluorochromes having one or both of a delectably different excitation and emission spectrum.

16. The method of claim 15, wherein the first fluorochrome is R-phycoerythrin, the second fluorochrome is allophycocyanin, the third fluorochrome is a tandem fluorochrome is a R-phycoerythrin-Cy5 tandem fluorochrome and the fourth fluorochrome is fluorescein isothiocyanate.

17. The method of claim 14, wherein the first fluorochrome is R-phycoerythrin and the second fluorochrome is allophycocyanin.

18. The method of claim 10, wherein the graft is a liver allograft.

19. A set of binding reagents for use in identifying tolerance in a graft recipient, comprising binding reagents permitting quantitation of pDC1 cells and pDC2 cells in the peripheral blood of a graft recipient.

20. The set of binding reagents of claim 19, wherein the binding reagents are antibodies.

21. The set of binding reagents of claim 19, wherein the binding reagents are selected to bind independently to HLA-DR, lin, CD123 and CD11c cellular markers, wherein lin consists of CD3, CD14, CD19 and CD20 markers.

22. The set of binding reagents of claim 21, wherein the binding reagents are anti-HLA-DR, anti-lin, anti-CD123 and anti-CD11c antibodies, each independently attached to a fluorochrome detectably different from the other fluorochromes.

23. The set of binding reagents of claim 21, wherein the binding reagents are anti-HLA-DR antibody conjugated to an R-phycoerythrin-Cy5 tandem fluorochrome, FITC-conjugated anti-lin antibody, R-phycoerythrin-conjugated anti-CD123 antibody and allophycocyanin-conjugated anti-CD11c antibody.

24. A method of identifying tolerance in a liver allograft recipient, comprising the step of obtaining a ratio of HLA-DR⁺lin⁻CD 123⁺CD11c⁻ cells and HLA-DR⁺lin⁻CD123^{lo}CD11c⁺ cells in a peripheral blood sample of the recipient and determining if the ratio is greater than a value indicative of tolerance in the recipient, wherein lin⁻ refers to the CD3⁻CD14⁻CD19⁻CD20⁻ phenotype.
